(19) United States
(12) Patent Application Publication
(10) Pub. No.: US 2011/0098790 A1
(43) Pub. Date: Apr. 28, 2011

(54) Methods for treating corneal disease

(76) Inventor: Albert DAXER, Linz (AT)

(21) Appl. No.: 12/925,162

(22) Filed: Oct. 14, 2010

Related U.S. Application Data
(60) Provisional application No. 61/279,824, filed on Oct. 26, 2009.

(51) Int. Cl. A61F 9/007

(52) U.S. Cl. 607/88

(57) ABSTRACT

A method for treating corneal disease includes the steps of forming a corneal pocket in a cornea at a depth from a corneal surface, introducing a corneal stiffening substance into the corneal pocket and irradiating the cornea with electromagnetic radiation.

### CROSS REFERENCE TO RELATED APPLICATIONS

This application claims priority under 35 U.S.C. §119(e)(i) and the benefit of co-pending U.S. Provisional Application No. 61/279,824 entitled “Method to treat corneal diseases such as keratoconus” filed on Oct. 26, 2009, which is incorporated in its entirety by reference herein.

### BACKGROUND OF THE INVENTION

1. Field of the Invention

The invention relates to methods for treating corneal disease. In particular, the invention relates to methods for treating corneal disease, such as keratoconus, by forming a corneal pocket in the cornea, introducing a corneal stiffening substance into the corneal pocket and irradiating the cornea with electromagnetic radiation.

2. The Prior Art

The cornea is the transparent, projecting outer “wall” of the eye. The optical quality of the cornea is of particular importance for the visual function of the eye. The outer corneal surface, which forms the interface between the cornea and the surrounding air, is responsible for approximately two thirds of the total dioptic power of the eye. Accordingly, a regular shape of the cornea is required to achieve good quality of vision.

There are several corneal diseases affecting the regularity of the cornea and reducing the visual function of the eye. Among these diseases, Keratoconus is one of the most prominent. The majority of these corneal diseases exhibit a progressive character, wherein the degree of corneal irregularity and visual dysfunction increases over time.

Seiler and his coworkers have developed a method of corneal cross-linking for reducing the irregularity of the cornea and stopping the progression of the disease, as described, for example, in Spörl E, Huhle M, Kasper M, Seiler T. (1997) Erhöhung der Festigkeit der Hornhaut durch Vernetzung. Ophthalmologe; and in Schnitzler E, Spörl E, Seiler T. (2000) Bestrahlung der Hornhaut mit UV-Licht and Riboflavingabe als neuer Behandlungsversuch bei einschmelzenden Hornhautprozessen, erste Ergebnisse bei 4 Patienten. Klin Monatsblatt Augenheilkunde. This therapeutic procedure of corneal cross-linking includes the removal of the epithelium of the cornea and the application of eye drops of riboflavin at the deepithelialiszed corneal surface for thirty minutes and consecutive ultraviolet (UV) irradiation of the cornea for another thirty minutes.

The application of riboflavin eye drops and UV irradiation without epithelial removal was shown to be clinically ineffective. The removal of the epithelium, however, results in severe pain and photophobia for several days after surgery. Moreover, although the known method is effective in stopping the progression of the corneal disease, the ability of the known method to cure the irregularity of the diseased cornea is very limited.

Accordingly, there exists a need for an effective method for treating corneal disease which preserves the epithelium. Moreover, a need exists for a painless method for stopping the progression of corneal disease which method provides the option of effectively correcting even higher grades of corneal irregularities than can be corrected using prior known methods.

### SUMMARY OF THE INVENTION

The invention relates to methods for treating corneal disease. In particular, the invention relates to methods for treating corneal disease, such as keratoconus, by forming a corneal pocket in the cornea, introducing a corneal stiffening substance into the corneal pocket and irradiating the cornea with electromagnetic radiation.

In one aspect of the invention, a method for treating corneal disease includes the steps of forming a corneal pocket in a cornea at a depth from a corneal surface, introducing a corneal stiffening substance into the corneal pocket and irradiating the cornea with electromagnetic radiation.

In further aspects of the invention, the corneal pocket may be formed at a depth of between approximately fifty microns and four hundred fifty microns from the corneal surface, preferably between approximately two hundred fifty microns and three hundred fifty microns from the corneal surface, and more preferably approximately three hundred microns from the corneal surface.

In a further aspect of the invention, the corneal pocket may have a diameter of between approximately two millimeters and ten millimeters.

In a further aspect of the invention, the step of forming a corneal pocket may includes forming a tunnel-like entry through the corneal surface. The tunnel-like entry may have a width of less than approximately six millimeters and preferably less than approximately five millimeters. The corneal stiffening substance may be introduced into the corneal pocket thorough the tunnel-like entry.

In further aspects of the invention, the corneal pocket is formed using a manual dissector, a mechanical microkeratome or a laser.

In a further aspect of the invention, the cornea may be irradiated with ultraviolet light, preferably ultraviolet A light.

In a further aspect of the invention, the cornea may be irradiated with electromagnetic radiation for less than thirty minutes. The cornea may be irradiated with electromagnetic radiation without removing the epithelium of the cornea.

In a further aspect of the invention, the method for treating corneal disease may additionally include the step of inserting a corneal implant into the corneal pocket. The corneal implant may be inserted into the corneal pocket thorough the tunnel-like entry. The corneal implant may be a continuous ring implant, a split ring implant and/or a compressible implant.

In a further aspect of the invention, the corneal implant may be inserted into the corneal pocket before the corneal stiffening substance is introduced into the corneal pocket and before the cornea is irradiated with electromagnetic radiation.

In a further aspect of the invention, the corneal implant may be inserted into the corneal pocket after the corneal stiffening substance is introduced into the corneal pocket and before the cornea is irradiated with electromagnetic radiation.

In a further aspect of the invention, the corneal implant may be inserted into the corneal pocket after the corneal stiffening substance is introduced into the corneal pocket and after the cornea is irradiated with electromagnetic radiation.

An advantage of a method for treating corneal disease according to an aspect of the invention is that the epithelium of the cornea is preserved. A further advantage of a method for treating corneal disease according to an aspect of the invention is that a painless method for stopping the progression of corneal disease is provided. A further advantage of a method for treating corneal disease according to an aspect of the invention is that the method provides the option of effectively correcting even higher grades of corneal irregularities than can be corrected using prior known methods.

### BRIEF DESCRIPTION OF THE DRAWINGS

Other benefits and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a cross section of a cornea with a corneal pocket formed therein for introduction of a corneal stiffening substance in a method according to an aspect of the invention;

FIG. 2 shows a top view of the cornea illustrated in FIG. 1;

FIGS. 3a through 3c show a top view of the direction of the cuts drawn by a blade in forming a corneal pocket in a method according to an aspect of the invention;

FIG. 3d shows a top view of a cornea with a corneal pocket produced by the cuts illustrated in FIGS. 3a through 3c;

FIG. 4 shows a side view in partial section of an exemplary device for forming a corneal pocket in a cornea in a method according to an aspect of the invention;

FIG. 5 shows a side view in partial section of a second exemplary device for forming a corneal pocket in a cornea in a method according to an aspect of the invention;

FIG. 6 shows a perspective view of a continuous ring corneal implant for insertion into a corneal pocket in a method according to an aspect of the invention;

FIG. 7 shows a perspective view of a split ring corneal implant for insertion into a corneal pocket in a method according to an aspect of the invention; and

FIG. 8 shows a cross-section of a cornea with a corneal implant for insertion into a corneal pocket in a method according to an aspect of the invention.

### DETAILED DESCRIPTION OF THE DRAWINGS

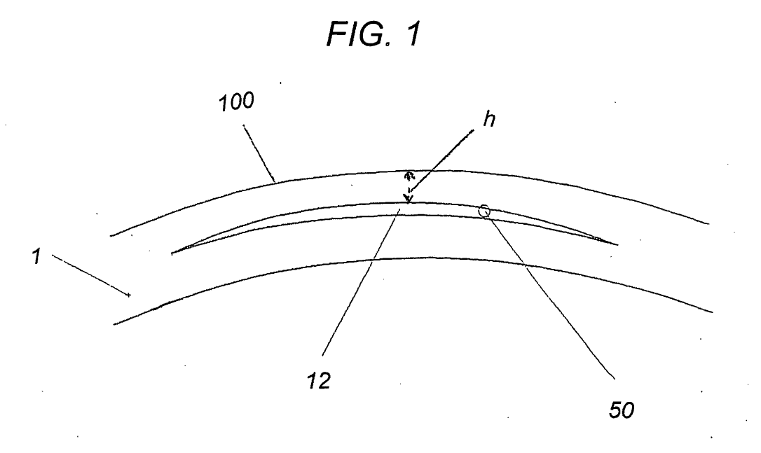

A method is provided for the treatment of corneal diseases, such as for example, Keratoconus. As illustrated in FIG. 1, a method for treating corneal disease according to an aspect of the invention includes the step of forming a corneal pocket 12 in a cornea 1 at a depth h from a corneal surface 100.

Corneal pocket 12 may be any suitable shape and/or size and is preferably a complete or incomplete laminar dissection which is roughly or virtually parallel to the front or back surface of the cornea 1. The corneal pocket 12 should be centered at least roughly with respect to the optical or anatomical axis. Alternatively, the corneal pocket 12 may be positioned off-center.

The depth of the corneal pocket 12 measured from the corneal surface 100 should be at least approximately fifty microns and less than approximately four hundred fifty microns. Thus, in a method according to an aspect of the invention, the corneal pocket 12 may be formed at a depth of between approximately fifty microns and four hundred fifty microns from the surface 100 of the cornea 1. In particular, corneal pocket 12 may be formed at a depth of between approximately two hundred fifty microns and three hundred fifty microns from the corneal surface 100. More particularly, corneal pocket 12 may be formed at a depth of approximately three hundred microns from the corneal surface 100.

Corneal pocket 12 is formed to have a sufficient diameter or extension D. The diameter D of the corneal pocket 12 should be less than approximately ten millimeters and larger than approximately six millimeters, preferably more than approximately seven millimeters and more preferably more than approximately eight millimeters. For example, the corneal pocket 12 may have a diameter D of approximately nine millimeters. In other embodiments, the corneal pocket 12 may have a diameter even smaller than approximately six millimeters, and in particular even less than approximately two millimeters. For example, in a method according to an aspect of the invention, corneal pocket 12 may be formed to have a diameter D of between approximately two millimeters and ten millimeters.

The corneal pocket 12 can be a closed pocket or a pocket with an opening which may be very small, for example an incision, or an opening which extends over several clock hours. Preferably, the corneal pocket 12 is closed along its entire circumference with the optional exception of a narrow tunnel-like entry or small pocket entry.

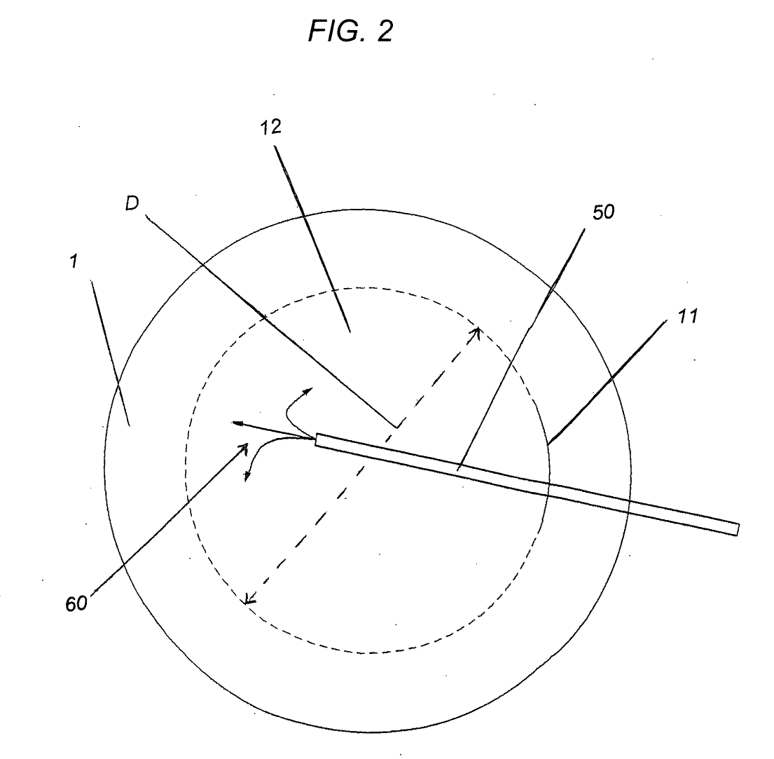

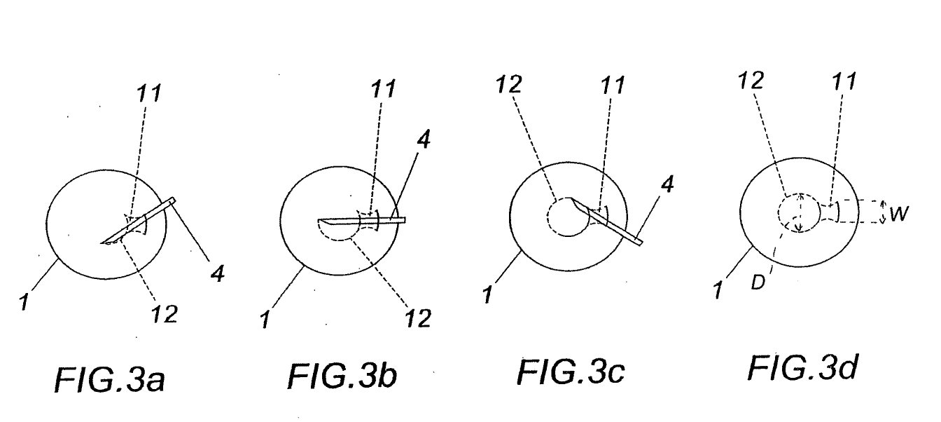

As illustrated, for example in FIGS. 2 and 3a-3d, the step of forming a corneal pocket 12 may include forming a tunnel-like entry or small pocket entry 11 through the corneal surface. The tunnel-like entry 11 may be narrow in width. For example, the width W of the tunnel-like entry 11 may be less than approximately six millimeters, preferably less than approximately five millimeters and more preferably less than approximately four millimeters. The entry opening of the corneal pocket 12 may, however, be of any other suitable size.

Formation of the corneal pocket 12 may be accomplished using a number of techniques and devices. For example, a mechanical microkeratome may be used to form the corneal pocket 12. Alternatively, laser cutting (using, for example a Femtosecond laser) may be used for form the corneal pocket 12. Manual dissection using a suitable manual dissector (such as, for example a crescent knife) is also possible; however, this technique has a much higher degree of difficulty and level of risk.

Exemplary devices suitable for forming a corneal pocket 12 in a method according to an aspect of the invention are described, for example, in applicant's co-pending U.S. patent application Ser. No. 10/555,353, the disclosure of which is hereby incorporated by reference in its entirety.

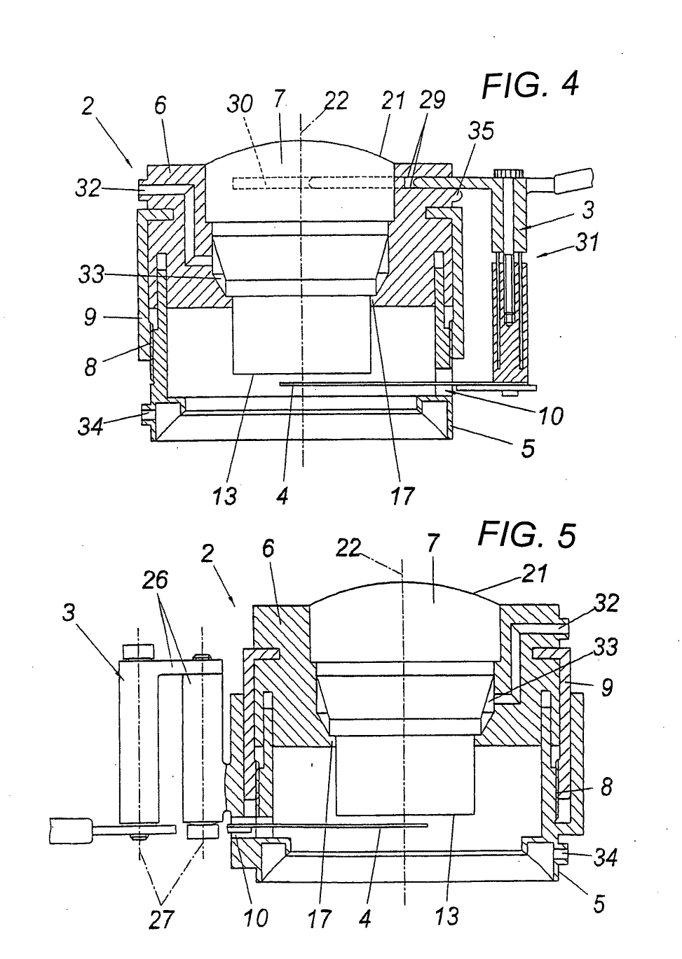

For example, FIGS. 4 and 5 show exemplary devices for forming a corneal pocket 12 in a cornea 1 in a method according to an aspect of the invention.

As shown, the exemplary devices for forming a corneal pocket 12 in a cornea 1 of an eye generally comprise a frame 2 and a holding device 3 for supporting a blade 4. The frame 2 has a fixation ring 5, which may be drawn onto the eye, and a receptacle 6, which may be coaxially displaced relative to the fixation ring 5 and which serves to accommodate an applanator 7 for deforming the cornea within the fixation ring 5. The cornea 1 thus projects through the fixation ring 5, within which, in particular offset in height relative to the fixation ring 5, the applanator 7 for impingement of the cornea is located. The fixation ring 5 is furnished with a thread 8 for coaxial displacement. A nut 9 mounted on the receptacle 6 engages the thread 8, thereby allowing for rotation.

By rotating the nut 9, the receptacle 6 and/or the applanator 7 may thus be displaced relative to the fixation ring 5 and/or the cornea 1. The holding device 3 for supporting the blade 4 is guided on the frame 2 in a plane that is perpendicular to the axis of the fixation ring 5, and the blade 4 passes through the frame 2 with clearance via a peripheral recess 10 and is mounted in front of the applanator 7. The blade 4 is, in particular, guided by the holding device 3 in such a way that said blade 4 is radially displaceable relative to the fixation ring 5 as well as movable around an axis perpendicular to the guiding plane via the holding device 3, for the purpose of cutting a pocket 12 through a merely tunnel-like entry 11 into the corneal tissue, as may in particular be inferred from FIGS. 3a through 3c.

Once the applanator 7 has been placed on the cornea 1 accordingly, the tip of the blade 4 is placed on cornea 1 and the outer tissue layers of the cornea 1 are penetrated in order to produce a tunnel-like entry 11. It is critical that the blade 4 does not slip from where it is placed on the corneal surface. Accordingly, the blade 4 passes through the frame recess 10 with clearance, and the holding device 3 supports a vibrator for setting the blade 4 in oscillatory motion in the cutting plane. The vibrator may be designed as a piezo element, which vibrates in the cutting plane of the blade 4, or as an unbalanced motor.

The receptacle 6 accommodating the applanator 7 may be designed as a receptacle for the stop-delimited receipt of interchangeable applanators 7 with differently curved contact faces 13 for applanation of the cornea. A stop 17 is illustrated in FIGS. 4 and 5. By using an applanator 7 with a specifically curved contact face 13 and determining a specific cutting depth, the radius of curvature of the pocket 12 to be produced may be accurately defined.

The applanators 7 are preferably made of transparent material, such as plastic or glass, and are designed as enlargement lenses 21 as illustrated in FIGS. 4 and 5, with their focal point lying in the area of the contact face 13, preferably on the axis of symmetry of the applanator 22. An applanator 7 being designed in such a way makes it relatively easy for a surgeon to monitor the progress of treatment of the cornea 1.

According to the exemplary embodiment illustrated in FIG. 5, the holding device 3 consists of a lever system comprising at least two lever arms 26 having pivot axes 27 which are perpendicular to the cutting plane of the blade 4; one lever arm 26 receives the blade 4 and the other lever arm 26 is linked to the frame 2, preferably to the receptacle 6.

According to the exemplary embodiment illustrated in FIG. 4, the holding device 3 may also comprise a forked blade guide receiving the blade 4, which is guided, possibly without clearance, between parallel faces 29 of a peripheral groove 30 provided on the frame 2, in particular on the receptacle 6. The blade 4 is offset to the forked blade guide, with the distance between the cutting plane of the blade 4 and the contact face 13 of the applanator 7 being adjustable by means of a position adjuster 31 having the shape of a winding gear. A knob 35 is provided on the receptacle 6 to allow the forked blade guide to be easily pushed in.

The applanator 7 may be fixed into the receptacle 6 by means of a partial vacuum. For this purpose, air may be sucked out of a chamber that is located between the receptacle and the applanator through a line 32. The applanator has the shape of a truncated cone, which allows easy insertion of the applanator. Furthermore, it is conceivable that other mechanical holding devices instead of the pressure line are used, including bayonet closures, magnetic, electromagnetic, hydraulic, or other equivalent mechanisms. A similar procedure is conducted when drawing the fixation ring 5 onto the eye by a pressure line 34.

As illustrated in FIG. 2, a method for treating corneal disease according to an aspect of the invention further includes the step of introducing or instilling a corneal stiffening agent or substance 60 into the corneal pocket 12. The introduction of the corneal stiffening substance 60 into the corneal pocket 12 may be performed via an appropriate cannula 50 and a syringe.

The corneal stiffening substance 60 supports the stiffening of corneal tissue and as a result stops the progression of the diseases in question. For example, the corneal stiffening substance 60 may promote cross-linking of the corneal tissue. The corneal stiffening substance may also have the ability to change the shape of the cornea immediately or over time.

The corneal stiffening substance or agent 60 may be a solid or a gel, but preferably is a liquid. The corneal stiffening substance 60 may be introduced into the corneal pocket 12 through the tunnel-like entry or incision 11, although it is also possible to inject the corneal stiffening substance 60 by means of a cannula via the walls of the corneal pocket 12.

The corneal stiffening substance 60 can be a pure or diluted substance. The amount of corneal stiffening substance applied to the cornea may be between approximately 0.1 milliliters and 10 milliliters, and is preferably approximately 3 milliliters. Penetration of the corneal stiffening substance 60 into the corneal tissue may be achieved either by rinsing, flushing or irrigating the corneal pocket 12 over a limited period of time with the corneal stiffening substance 60. Another way of administering the corneal stiffening substance 60 is to create a depot of the corneal stiffening substance 60 within the corneal pocket 12 over a limited period of time. The associated period of time for applying or administering the corneal stiffening substance 60 via the corneal pocket may be thirty minutes or less, is preferably less than fifteen minutes, and is more preferably less than five minutes. For example, in a method according to an aspect of the invention, the corneal stiffening substance 60 may be applied or administered over a time period of approximately two to three minutes.

One exemplary corneal stiffening substance 60 for introduction into the corneal pocket 12 is riboflavin. For example, in a method according to an aspect of the invention, the corneal stiffening substance 60 includes 0.1% riboflavin diluted in a 20% dextran 500 solution. In general, the corneal stiffening substance 60 may be hypotonic, isotonic or hypertonic or hypoosmolar, isoosmolar or hyperosmolar and can be combined with any suitable diluent. The active substance which may be diluted or pure may be any suitable corneal stiffening substance or composition of substances.

A method for treating corneal disease according to an aspect of the invention further includes the step of irradiating the cornea with electromagnetic radiation. The electromagnetic radiation may comprise, for example ultraviolet (UV) light and more particularly may comprise ultraviolet-A (UV-A) light. For example, the irradiation of the cornea may be performed using a UV-A light source providing an irradiation of the cornea at approximately 340 to 380 nanometers wavelength (such as, for example, 365 nanometers) with an intensity between approximately 0.1 milliwatts/square centimeter and 20 milliwatts/square centimeter (such as, for example, 3 milliwatts/square centimeter) over a limited area at the corneal surface between approximately 3 square millimeters and 120 square millimeters, preferably between approximately 70 square millimeters and 100 square millimeters, corresponding roughly to an irradiated diameter at the corneal surface of approximately 9 to 11 millimeters. The irradiated zone may be centered or off-center.

The duration of the irradiation of the cornea may be less than one hour. In particular, in a method according to an aspect of the invention, the cornea is irradiated with electromagnetic radiation for less than thirty minutes and more particularly for less than fifteen minutes.

The irradiation of the cornea with electromagnetic radiation should preferably be performed after the introduction of the corneal stiffening substance 60 into the corneal pocket 12 as described above. In a method according to an aspect of the invention, the step of irradiating the cornea 1 with electromagnetic radiation is performed without removing the epithelium of the cornea. In particular, the irradiation of the cornea 1 may be performed without removing or substantially altering the epithelium either before or during the irradiation process.

A method for treating corneal disease according to an aspect of the invention may optionally includes the further step of inserting a corneal implant 71, 72, 73 into the corneal pocket 12. In particular, the corneal implant 71, 72, 73 may be inserted into the corneal pocket 12 through the tunnel-like entry 11.

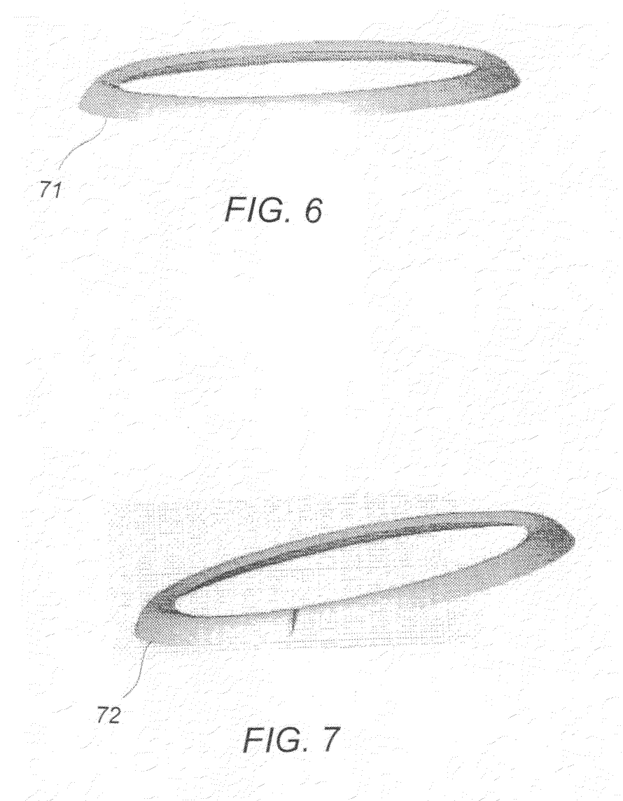

The corneal implant may be any implant suitable to correct a refractive error. For example, the corneal implant may be a continuous ring implant 71, as illustrated in FIG. 6 or a split ring implant 72, as illustrated in FIG. 7. The corneal implant may comprise a compressible implant.

Exemplary corneal ring implants suitable for inserting into a corneal pocket in a method according to an aspect of the invention and methods of their use are described, for example, in applicant's co-pending U.S. patent application Ser. No. 12/224,966, the disclosure of which is hereby incorporated by reference in its entirety.

Additional exemplary corneal implants suitable for inserting into a corneal pocket in a method according to an aspect of the invention and methods of their use are described, for example, in applicant's co-pending U.S. patent application Ser. No. 12/227,533, the disclosure of which is hereby incorporated by reference in its entirety.

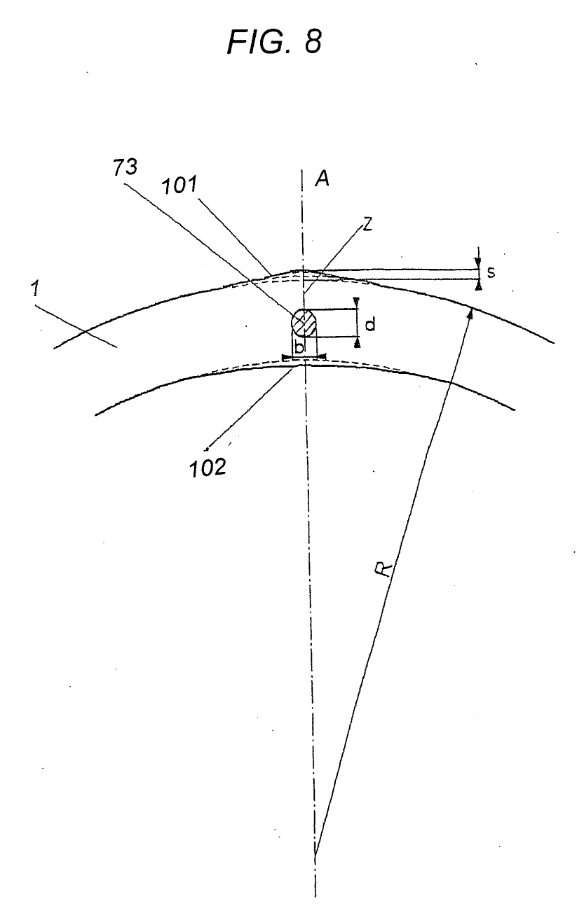

For example, FIG. 8 shows a cross-section of a cornea 1 with a corneal implant 73 for insertion into a corneal pocket 12 in a method according to an aspect of the invention. In particular, FIG. 8 shows a cross section of the cornea 1 of a human eye with a radius of curvature R including an optical center Z. A corneal implant 73 is implanted in the corneal tissue of the cornea 1, having an effective thickness d of more than 50 microns, measured in the direction of the optical axis A of the eye, and a width b of less than 1 millimeters, measured in a plane perpendicular to the direction of thickness. The implant 73 may also be placed off-center.

Corneal implant 73 has no imaging function in relation to the human eye, which means that the light rays entering the eye are not focused on the retina (not depicted in the drawings) of the eye due to the optical properties of the corneal implant 73. Instead, the implantation of the corneal implant 73 results in a central volume addition and thus in an aspherical surface contour 101 of the cornea 1 around the optical center Z of the cornea 1, which also facilitates multi-focal imaging. In a particular embodiment, the implant 73 may have an optical function, for example, as a lens or pinhole.

In contrast to the known state-of-the-art corneal implants and vision correction methods, corneal implant 73 deliberately lacks an optical function and may be introduced into the optical center Z of the eye. In particular, corneal implant 73 serves to correct the impaired vision exclusively by altering the curvature R of the cornea 1 around the corneal implant 73. Although this arrangement may lead to deformations in the area of the corneal back face 102, these are of only minor relevance for vision correction.

Corneal implant 73 may be of any type of transparency; for example it may be fully opaque, semi-transparent, or fully transparent. Moreover, because corneal implant 73 has no imaging function in relation to the eye, it may be of any color whatsoever, preferably black to assure compatibility with the black pupil.

In a method according to an aspect of the invention, the step of inserting the corneal implant 71, 72, 73 may be performed after the step of creating the corneal pocket 12, after the step of introducing the corneal stiffening substance 60 into the corneal pocket 12 and after the step of irradiating the cornea 1 with electromagnetic radiation. Thus, the step of inserting the corneal implant 71, 72, 73 may be performed as a fourth step (i.e., after creation of the corneal pocket 12, after introduction of the corneal stiffening substance 60 into the corneal pocket 12 and after irradiation of the cornea 1).

In a method according to another aspect of the invention, the step of inserting the corneal implant 71, 72, 73 may be performed after the step of creating the corneal pocket 12, after the step of introducing the corneal stiffening substance 60 into the corneal pocket 12 and before the step of irradiating the cornea 1 with electromagnetic radiation. Thus, the step of inserting the corneal implant 71, 72, 73 may be performed as a third step (i.e., after creation of the corneal pocket 12, after introduction of the corneal stiffening substance 60 into the corneal pocket 12 and before irradiation of the cornea 1).

In a method according to another aspect of the invention, the step of inserting the corneal implant 71, 72, 73 may be performed after the step of creating the corneal pocket 12, before the step of introducing the corneal stiffening substance 60 into the corneal pocket 12 and before the step of irradiating the cornea 1 with electromagnetic radiation. Thus, the step of inserting the corneal implant 71, 72, 73 may be performed as a second step (i.e., after creation of the corneal pocket 12, before introduction of the corneal stiffening substance 60 into the corneal pocket 12 and before irradiation of the cornea 1).

In this embodiment, the creation of a depot of the cornea stiffening substance 60 within the corneal pocket 12 over a limited period of time is very easy if a continuous ring implant is inserted and if the corneal stiffening substance 60 is introduced “inside the ring” and the depot is delimited by the ring implant. In this case, the anterior and posterior lamellae of the cornea (for example, the anterior and posterior wall of the corneal pocket) as well as the ring implant itself capture or delimit the depot of corneal stiffening substance 60 in the pocket and prevents the corneal stiffening substance 60 from leaking (for example, flowing via the entry to outside the corneal tissue instead of diffusing into the tissue via the pocket walls).

In a method according to an aspect of the invention, a suitable corneal implant may be either centered on the optical axis or the anatomical axis of the cornea of the eye, or alternatively positioned off-center with respect to the optical axis or the anatomical axis of the cornea of the eye.

Accordingly, while a number of embodiments of the present method have been shown and described, it is obvious that many changes and modifications may be made thereunto without departing from the spirit and scope of the invention.

What is claimed is:

1. A method for treating corneal disease, the method comprising the steps of:
a) forming a corneal pocket in a cornea at a depth from a corneal surface;
b) introducing a corneal stiffening substance into the corneal pocket; and
c) irradiating the cornea with electromagnetic radiation.

2. The method for treating corneal disease according to claim 1, wherein the step of forming a corneal pocket comprises forming the corneal pocket at a depth of between approximately fifty microns and four hundred fifty microns from the corneal surface.

3. The method for treating corneal disease according to claim 2, wherein the step of forming a corneal pocket comprises forming the corneal pocket at a depth of between approximately two hundred fifty microns and three hundred fifty microns from the corneal surface.

4. The method for treating corneal disease according to claim 3, wherein the step of forming a corneal pocket comprises forming the corneal pocket at a depth of approximately three hundred microns from the corneal surface.

5. The method for treating corneal disease according to claim 1, wherein the step of forming a corneal pocket comprises forming the corneal pocket to have a diameter of between approximately two millimeters and ten millimeters.

6. The method for treating corneal disease according to claim 1, wherein the step of forming a corneal pocket comprises forming a tunnel-like entry through the corneal surface.

7. The method for treating corneal disease according to claim 6, wherein the step of forming a tunnel-like entry through the corneal surface comprises forming the tunnel-like entry to have a width of less than approximately six millimeters.

8. The method for treating corneal disease according to claim 7, wherein the step of forming a tunnel-like entry through the corneal surface comprises forming the tunnel-like entry to have a width of less than approximately five millimeters.

9. The method for treating corneal disease according to to claim 6, wherein the step of introducing a corneal stiffening substance into the corneal pocket comprises introducing the corneal stiffening substance into the corneal pocket through the tunnel-like entry.

10. The method for treating corneal disease according to claim 1, wherein the step of forming a corneal pocket comprises using a manual dissector to form the corneal pocket.

11. The method for treating corneal disease according to claim 1, wherein the step of forming a corneal pocket comprises using a mechanical microkeratome to form the corneal pocket.

12. The method for treating corneal disease according to claim 1, wherein the step of forming a corneal pocket comprises using a laser to form the corneal pocket.

13. The method for treating corneal disease according to claim 1, wherein the step of irradiating the cornea with electromagnetic radiation comprises irradiating the cornea with ultraviolet light.

14. The method for treating corneal disease according to claim 13, wherein the step of irradiating the cornea with electromagnetic radiation comprises irradiating the cornea with ultraviolet A light.

15. The method for treating corneal disease according to claim 1, wherein the step of irradiating the cornea with electromagnetic radiation comprises irradiating the cornea with electromagnetic radiation for less than thirty minutes.

16. The method for treating corneal disease according to claim 1, wherein the step of irradiating the cornea with electromagnetic radiation is performed without removing the epithelium of the cornea.

17. The method for treating corneal disease according to claim 1, further comprising the step of inserting a corneal implant into the corneal pocket.

18. The method for treating corneal disease according to claim 17, wherein the step of forming a corneal pocket comprises forming a tunnel-like entry through the corneal surface and wherein the step of inserting a corneal implant into the corneal pocket comprises inserting the corneal implant through the tunnel-like entry.

19. The method for treating corneal disease according to claim 17, wherein the step of inserting a corneal implant into the corneal pocket comprises inserting a continuous ring implant.

20. The method for treating corneal disease according to claim 17, wherein the step of inserting a corneal implant into the corneal pocket comprises inserting a split ring implant.

21. The method for treating corneal disease according to claim 17, wherein the step of inserting a corneal implant into the corneal pocket comprises inserting a compressible implant.

22. The method for treating corneal disease according to claim 17, wherein the step of inserting a corneal implant into the corneal pocket is performed before the step of introducing a corneal stiffening substance into the corneal pocket and before the step of irradiating the cornea with electromagnetic radiation.

23. The method for treating corneal disease according to claim 17, wherein the step of inserting a corneal implant into the corneal pocket is performed after the step of introducing a corneal stiffening substance into the corneal pocket and before the step of irradiating the cornea with electromagnetic radiation.

24. The method for treating corneal disease according to claim 17, wherein the step of inserting a corneal implant into the corneal pocket is performed after the step of introducing a corneal stiffening substance into the corneal pocket and after the step of irradiating the cornea with electromagnetic radiation.
